(19) United States
(12) Patent Application Publication
(10) Pub. No.: US 2008/0057111 A1
(43) Pub. Date: Mar. 6, 2008

(54) Herbal composition for flu prevention and treatment

(76) Inventor: Lixian Jiang, Land O Lakes, FL (US)

Correspondence Address: LIXIAN JIANG, 2102 Camp Indianhead Road, Land O Lakes, FL, 34639, US

(21) Appl. No.: 11/511,788

(22) Filed: Aug. 29, 2006

(51) Int. Cl. A61K 36/315; A61K 36/195; A61P 31/16

(52) U.S. Cl. 424/440; 424/773; 424/774

(57) ABSTRACT

A herbal formulation for prevention and/or treatment of flu. The formulations comprise of plant or plant extract together with vitamins and conventional additives. The plant is isatis indigotica, or strobilanthes cusia or the mixture thereof The formulations are in the form of tablet, powder, capsule, liquid or gum. Preferably, the formulations are in the manner of effervescent or chewing gum.

### FIELD OF THE INVENTION

The present invention is in the field of herbal composition to prevent and /or treat flu. The present composition consists of isatis indigotia, strobilanthes cusia and other conventional addictives, and is in the form of powder, tablet, capsule, liquid or and gum. Preferably, this composition is effervescent or in the form of chewing gum.

### BACKGROUND OF THE INVENTION

Flu is a pandemic disease in human mostly caused by influenza, a RNA virus belonging to orthomyxoviridae family. Once infected, the patients have characteristic symptoms of respiratory disease such as fever, pharyngitis (sore throat), myalgia (muscle pains), sever headache, coughing, and malaise (weakness and fatigue). In some cases, flu can also be caused by bacteria, and has a similar symptom.

In history, flu killed millions of people. For example, in 1918-1919, one mutant virus strain of influenza A, called Spanish flu, killed 50 to 100 million people in just one year. It is estimated that influenza can easily kill 25 million people in 6 weeks in one outbreak. In contrast, it takes 6 years for AIDS to kill the same amount of people. In United States, around 28% population suffered this disease, and 500,000 of 675,000 died. The large scale of infected population is due to its air borne spreading and fast mutation.

As of the treatment, antibiotic does not work on common flu, since the common flu is caused by virus. Currently there are some antivirus medications on market claiming treat flu virus, but their effects are limited. Most of these medications are based on alleviation of symptoms, such as decrease fever, sooth throat, and lessen headache. The recovery basically relies on patient's own immune system. With flu duration, the immune system could produce target antibody to clean the virus, and prevent patients from second infection. The process usually takes 7 days. Therefore, if the patients keep a good hygiene habit, and strong enough, even without treatment patients can get recovery after 7 days. However, for the weak population such as kids or aged people, if the symptoms are not under control, it is easy to take patients' life.

Another effective method to prevent flu spreading and against flu infection is to take some influenza vaccines in pandemic season. Currently, vaccines are engineered biomaterials under the regulation of FDA in Unite States. Vaccines are to stimulate immune system to produce target antibody, which functions as a guard line against flu infection. No matter how long the antibody exists after the flu shot, people still need to get another flu shot in the next flu season, since influenza mutates very fast, and almost every year there are different influenza strains occurrence. Currently, WHO and other health organization predict and report the possibility of the new mutant virus yearly, and direct manufacture to produce new vaccines to meet market needs. However, even with this effort, there are still millions people around the world as well as in the United State suffering this disease. Therefore, it is necessary to find an economic alternative for prevention flu infection.

Isatis indigotica Fort is the root of Isatis indigotica, a biennial herbaceous plant widely distributed in Asia. The dried root, also called Ban-Lan-Gen, is a traditional Chinese herb, and it has been used in traditional Chinese medicine for hundreds of years as antipyretic, antiviral, anti-inflammatory, and anti-influenza agents. It has the effect to lessen toxicity, cool the blood and sooth the throat.

Strobilanthes cusia (Acanthaceae) is another biennial herbaceous plant, called Daqingye in traditional medicine Chinese. It has the similar effect as Isatis indigotica Fort, and has been used to treat flu patients in combination with Isatis indigotica Fort for many years. During the outbreak of severe acute respiratory syndrome (SARS) in 2003, both Daqingye and Banlangen were prescribed as a preventive herbaceous medication against SARS infection at that time. The practical application of these medications was successful, and their effects were further demonstrated in lab. Since then, they have become two of the most popular traditional Chinese medicines.

The antivirus effect of these two herb plants was further demonstrated with the standard western medical study. The effect of these two herb plants was tested respectively in vitro with bacterial endotoxin (ET) test, with influenza virus infected human cells, and in rodent animals. It was found that the herb extract has anti-endotexin activity, antivirus, antinociceptive, antiinflammatory and antipyretic effects. Further studies convinced that the extract of Isatis indigotica Fort or Strobilanthes cusia (Acanthaceae) is in rich of some organic acids including syringic acid, σ-aminobenzoic acid, salicylic acid, and benzoic acid. All of the organic acids have anti-endotoxin activity in vivo. Another component of the extract, indirubin, has the antivirus effect. Interestingly, recent report indicated that the components from the herb including indigo, indirubin, indican, sinigrin, beta-sitosterol, aloeemodin, hespertin and daidzein has the inhibitory effect on “severe acute respiratory syndrome (SARS)” virus.

However, all the past application of these herbs against flu in clinic is based on the raw materials of these herb plants such as dried root, dried leaves, or their primary extract. These medications are hard to be dissolved, and are not in good taste. This greatly limits its application. This invention discloses a convenient and flavoring formula for flu prevention and treatment.

### SUMMARY OF THE INVENTION

This patent discloses a formula with Chinese traditional herbs, comprising of the extract of isatis indigotica Fort and/or strobilanthes cusia, for flu prevention and/or treatment. In one aspect, the preparation composition includes vitamins C, E, conventional flavoring and sweetener. In one embodiment the composition could be in form of powder, tablet, liquid, capsule or gum. Preferably, the composition comprises of effervescent agent, which includes an acid and a base, wherein the composition disintegrates in water very fast. Preferably, the composition is in the form of chewing gum wherein a gum base is included.

When the first sign of cold, the composition is dropped in a cup of cool or hot water. Once the effervescent agent contacts water, the acid agent has a chemical reaction with the base agent with gas release, which disintegrates and disperses the other components of the composition into solution, and promotes composition dissolution.

In another embodiment, the composition could be chewed directly as a gum without any water or beverage. When the composition touches and mixes saliva, the composition is dissolved in saliva and absorbed through palate and digest gut. The effective of this composition has anti-flu and anti-virus effect, which prevent patients from infection of disease or promoting recovery.

### DETAILED DESCRIPTION OF THE INVENTION

The present herb composition is in form of powder, tablet, capsule, which comprises of at least one component, isatis indigotica Fort or its extract. The component takes about 10 to 90% of total weight of the composition. Alternatively, the composition could further comprise of strobilanthes cusia or its extract. Even the herb composition is not necessary to include “strobilanthes cusia extract”, it is preferred to include this indigent in the said herb composition.

Generally, the root of isatis indigotica or the leaf of strobilanthes cusia or herein of the mixture is selected for this preparation. The herb extract is prepared in disinfected facility. The herb plant is washed cleanly, and immersed in 95% aqueous alcoholic solution for 12-72 hours. The said alcoholic solution is ethanol. The mixture of the solution is refluxed at 50-80° C. for three times. The extracted solution is filtered and concentrated with vacuum evaporator. After these processes, the fined product is obtained. Please be alert! The said method for processing the extract is an example, and there is no intention to limit the methods for preparing the herb extract. To a skilled art in this area, the herb extract could be obtained through other methods.

In another embodiment, the composition further composes of vitamin C, which takes 1% to 15% of the total weight of the composition. In another embodiment, the composition also includes vitamin E, and it takes 1% to 15% of the total weight of the composition. The vitamins serve as nutrition supply to improve body immune system. In another embodiment, flavoring, sugar or other artificial sweeteners such as aspartame is added to adjust the flavor of this composition. These conventional addictives are about 01% to 5% of the total weight.

Preferably, the herb composition includes effervescent agents when it is in form of powder, tablet, or capsule. The effervescent agents contain an acid base and a basic base, and they are about 10% to 50% of total weight of the composition. The acid base is selected from the group consisting of an organic acid, for example a carboxylic acid, organic acid salts, organic acid anhydrides, inorganic acids, inorganic acid salts, and mixtures thereof. Preferably, the organic acid, and related salts and anhydrides are selected from carboxylic acids having up to 8 carbon atoms such as, for example acetic, formic, propionic, malic, tartaric, citric, glycolic, maleic, fumaric, adipic, succinic, lactic, gluconic, and butyric acids and their homologs, and sulphamic acid. Examples of inorganic acid or acid salts include boric acid, sodium dihydrogen phosphate, disodium dihydrogen pyrophosphate, and sodium acid sulfite. Among them, citrate or citric acid salt is preferred.

In this composition, the basic component is selected from a group consisting of carbonates, bicarbonates, sequicarbonates and mixtures thereof. Preferably, carbonates, bicarbonates or mixtures thereof are used to make this pharmaceutical composition. When the composition is dissolved into water, the acid base has a chemical reaction with basic base, leading to the production of carbon dioxides. The burst of gas production promotes the disintegration and dissolution of the composition.

Thus, the composition formula mainly consists of:
isatis indigotica Fort; strobilanthes cusia extract or wherein the mixture; vitamin C; vitamin E; sugar (or artificial sweetener), and effervescent agents. The pharmaceutical composition is plasticized into powder, tablet or capsule

### EXAMPLE A

One example of the pharmaceutical composition, wherein as example A, shows as below: Isatis indigotica Fort extract 50% (w/w); Vitamin C 6% (w/w); Vitamin E 3% (w/w); Monosodium citrate 20% (w/w); Sodium bicarbonate 20% (w/w), aspartame 1% (w/w).

In this example, isatis indigotica Fort extract serves as anti-flu and anti-virus agent. The vitamin C and E are nutrition supplement, and also service as acid base at wet condition. Since the extract of isatis indigotica Fort is slightly bitter, aspartame is added to adjust the flavor of the pharmaceutical composition as sweetener. The monosodium citrate and sodium bicarbonate serve as effervescent agents, and they also serve as binders to plasticize the above composition.

### EXAMPLE B

Another example of the pharmaceutical composition, wherein as example B consists of: Isatis indigotica Fort extract 25% (w/w); strobilanthes cusia extract 25% (w/w); Vitamin C 6% (w/w); Vitamin E 3% (w/w); Monosodium citrate 20% (w/w); Sodium bicarbonate 20% (w/w), aspartame 1% (w/w).

Different from example A, example B use isatis indigotica Fort extract and strobilanthes cusia extract as anti-flu and anti-virus agents instead of isatis indigotica Fort extract alone. The said two components share 50% weight of the total pharmaceutical composition, and each of them takes 25% total weight of the herb composition.

In one experiment, the herb composition A and B were diluted into distilled water at the final concentration of 10% (w/w). Sodium chloride solution served as control dose. Japanese rabbits were injected with endotoxin (ET) solution (20 EU/mL) at a dose of 2 ml/kg. Following that, animals were feed with herb composition A or composition B or sodium chloride solution randomly at the dose of 5 ml/kg. The temperature was measured every 0.5 hour for 4 hours. The body temperature in animals treated with herb composition A or herb composition B was significantly lower than animals treated with sodium chloride solution, while herb composition B is better than herb composition A with more animals in low body temperature.

In another study, Kunming strain mice were randomly divided into control, herb composition group A and herb composition B group. All animals were pretreated with lipopolysaccharides (LPS) at the dose of 0.2 mL/20 g. All herb compositions were dissolved into distilled water at final concentration of 10% (w/w), and fed animals at the 1 ml/20 g. The control animals were treated with sodium chloride with the same dose as the herb composition solution. The death rate of all mice was observed over 72 hours. The death rate is 70% in animals treated with sodium chloride, while the death rate in herb composition A treated group and composition B treated group was 30% and 20% respectively. The improvement is significantly in herb treated animals, and herb composition B is slightly better than herb composition A.

The anti-virus effect of herb composition A and B were studied in cultured human epithelial cells. The epithelial cells were infected with influenza virus B, and the cells were treated with composition A or composition B. In culture condition, the applied herb compositions were dissolved in DMEM medium, and applied in cultured cells at final concentration 1% (w/v). The cytokines including RANTES (regulated on activated normal T cell expression and secreted), MCP-1 (monocyte chemoattractant protein-1), MIP-1α (Macrophage inflammatory protein 1α) were studied. Both herb composition A and herb composition B had an inhibitory effect on cytokines production in influenza infected cells, and the inhibitory effect of herb composition A is stronger than herb composition B.

In addition, the time to dissolve herb composition A and herb composition B was studied in experiment. Once the composition touches the water, the effervescent agents have a chemical reaction, and carbon dioxides are released out to disintegrate the composition. As respected, there was no significant difference in carbon dioxide release in comparison with the two herb compositions, and it takes the same amount of time for these two herb composition of these two herb composition generally.

When the composition is in form of solution, the effervescent agents are not necessary to be included in this formula.

Preferably, the herb composition could be in form of gum, wherein gum base is a must, and takes about 5% to 95% total weight of the herb composition. The gum base generally comprises elastomer, resins, fats and oils, waxes, softeners and inorganic fillers. To a skilled art in this area, the elastomer may be polyisobutylene, isobutylene-isoprene copolymer and styrene butadiene rubber, as well as natural latexes such as chicle. Resins may include other materials such as polyvinylacetate and terpene resins. To give some example materials here just wants to clarify the definition of some science terms, and does not mean the limitation of the resource. To one skilled art in this area, there are more materials should be included as gum base.

In another embodiment, the herb composition further comprises of isatis indigotica Fort extract or strobilanthes cusia extract or therein mixture. The herb extract is about 10-90% of total weight. The ratio of isatis indigotica Fort extract and strobilanthes cusia extract is from 1:9 to 9:1, or the herb extract only, for example isatis indigotica Fort or strobilanthes cusia extract alone. In another embodiment, the herb composition also includes vitamin C, vitamin E, some flavoring and sweeteners. The sweeteners may be sugar, saccharin, aspartame, sucrlose, neotame, and acesulfame potassium et al. The flavorings may include a group of molecules such as glycin salt, guanylic acid salts, inosinic acid salts, 5′-ribonucleotides, malic salt and tartaric salt The listed sample materials do not mean the limitation of the resource, and to the knowledge of a killed art in this field, there are more substances in this category. The flavoring and sweeteners is about 0.1-5% of total weight. Vitamin C is in amount of about 1 to 15% of total weight, and vitamin E is in amount of about 1% to 15% of total weight.

In the process of produce, the gum base is melted to a soluble liquid. Herb extracts, vitamin C, vitamin E, some flavoring and sweeteners are slowly added to the gum base until the liquid cools down like dough. To a skilled art in this area, the dough is shaped, and cut into stick for package.

Please be alert that in the description of this patent, the numerical value with the word “about” or “proximally” in the specification just states the proximal range in this invention although the below and up value is indicated, unless otherwise it is specified. In this manner, a slight up or below the indicated value is allowed, and there is no significant difference in their effect with the formula specified in this specification and the slight modification. For example, when the range is about between 20% and 60%, a modification, for example 10% or 80%, is still allowed. In this disclosure, the range of the numerical value continuously covers all the number between the minimum and maximum values.

In addition, “such as”, “about”, “preferred”, “preferably” in this patent is to illustrate the content of the disclosure, there is no intention to limit the scope of the claims. In this patent, some examples are also intended to clarify the content of the dislosure. It dose not mean the limitation of scope of the claim in this patent.

What is claimed is:

1. An anti-virus or anti-bacteria herbal formula, the formula comprises of isatis indigotica Fort, or strobilanthes cusia or wherein the mixture;

2. A formula as claimed 1, wherein the isatis indigotica Fort, or strobilanthes cusia could be the herb extract;

3. A formula as claimed 1, the formula further comprises of vitamin C (1% to 15%, w/w), vitamin E (1% to 15%, w/w);

4. A formula as claimed 2, the formula further comprises of flavor (0.1% to 5% w/w), sweetener (0.1% to 5% w/w);

5. A formula as claimed 1, wherein isatis indigotica Fort, or strobilanthes cusia or the mixture is between 10% to 90% of the total weight of the composition;

6. A formula as claimed 2, wherein isatis indigotica Fort extract, or strobilanthes cusia extract or the mixture is between 10% to 90% of the total weight of the composition;

7. (canceled)

8. (canceled)

9. (canceled)

10. (canceled)

11. A formula as claimed 1, the composition is in the form of a tablet, a capsule, a powder, a liquid or a gum;

12. A formula as claimed 2, the composition is in the form of a tablet, a capsule, a powder, a liquid or a gum;

13. A formula as claimed 3, the composition is in the form of a tablet, a capsule, a powder, a liquid or a gum;

14. A formula as claimed 4, the composition is in the form of a tablet, a capsule, a powder, a liquid or a gum;

15. A formula as claimed 11, when the composition is in the form of a tablet, a capsule, a powder, the composition could further comprises of effervescent agents, which is about 10 to 50% of the total weight of the composition.

16. A formula as claimed 12, when the composition is in the form of a tablet, a capsule, a powder, the composition could further comprises of effervescent agents, which is about 10 to 50% of the total weight of the composition.

17. A formula as claimed 13, when the composition is in the form of a tablet, a capsule, a powder, the composition could further comprises of effervescent agents, which is about 10 to 50% of the total weight of the composition.

18. A formula as claimed 14, when the composition is in the form of a tablet, a capsule, a powder, the composition could further comprises of effervescent agents, which is about 10 to 50% of the total weight of the composition.

19. A formula as claimed 15, wherein the effervescent agents containing one acid base and one basic component. The acid base is selected from the group consisting of an organic acid, or it related organic salt. The basic component is selected from an organic base or it related organic salt, such as carbonates, bicarbonates, sequicarbonates and mixtures thereof.

20. A formula as claimed 16, wherein the effervescent agents containing one acid base and one basic component. The acid base is selected from the group consisting of an organic acid, or it related organic salt. The basic component is selected from an organic base or it related organic salt, such as carbonates, bicarbonates, sequicarbonates and mixtures thereof.

21. A formula as claimed 17, wherein the effervescent agents containing one acid base and one basic component. The acid base is selected from the group consisting of an organic acid, or it related organic salt. The basic component is selected from an organic base or it related organic salt, such as carbonates, bicarbonates, sequicarbonates and mixtures thereof.

22. A formula as claimed 18, wherein the effervescent agents containing one acid base and one basic component. The acid base is selected from the group consisting of an organic acid, or it related organic salt. The basic component is selected from an organic base or it related organic salt, such as carbonates, bicarbonates, sequicarbonates and mixtures thereof.

23. A formula as claimed 11, when the composition is in the form of a gum, the composition could further comprises of a gum base, which is about 5 to 95% of the total weight of the composition. The gum base generally comprises elastomer, resins, fats and oils, waxes, softeners, inorganic fillers or any other materials to a skilled art in this field.

24. A formula as claimed 12, when the composition is in the form of a gum, the composition could further comprises of a gum base, which is about 5 to 95% of the total weight of the composition. The gum base generally comprises elastomer, resins, fats and oils, waxes, softeners, inorganic fillers or any other materials to a skilled art in this field.

25. A formula as claimed 13, when the composition is in the form of a gum, the composition could further comprises of a gum base, which is about 5 to 95% of the total weight of the composition. The gum base generally comprises elastomer, resins, fats and oils, waxes, softeners, inorganic fillers or any other materials to a skilled art in this field.

26. A formula as claimed 14, when the composition is in the form of a gum, the composition could further comprises of a gum base, which is about 5 to 95% of the total weight of the composition. The gum base generally comprises elastomer, resins, fats and oils, waxes, softeners, inorganic fillers or any other materials to a skilled art in this field.
